(19) United States
(12) Plant Patent Application Publication
(10) Pub. No.: US 2023/0337561 P1
(43) Pub. Date: Oct. 19, 2023

(54) CANNABIS PLANT NAMED 'DRG1'

(50) ;

(71) Applicant: Michael Ray Fowler, McKinleyville, CA (US)

(72) Inventor: Michael Ray Fowler, McKinleyville, CA (US)

(21) Appl. No.: 18/445,208

(22) Filed: May 26, 2023

Related U.S. Application Data
(63) Continuation-in-part of application No. 17/369,814, filed on Jul. 7, 2021.
(60) Provisional application No. 63/049,563, filed on Jul. 8, 2020.

(51) Int. Cl. A01H 6/28

(52) U.S. Cl. PLT/258

(57) ABSTRACT

The unique annual herbaceous Cannabis plant variety ‘DRG1’ is provided. The variety can be distinguished by its outstanding features of phenotypic/morphologic features and phytochemical differences from parent and existing strains.

### CROSS REFERENCE TO RELATED APPLICATIONS

The present Application for Patent is a continuation in-part of U.S. application Ser. No. 17/369,814 filed on Jul. 7, 2021, which in turn claims the benefit of Provisional Application No. 63/049,563 entitled “CANNABIS PLANT NAMED ‘DRG1’” filed Jul. 8, 2020, and hereby expressly incorporated by reference herein.

Latin name of the genus and species: Genus—Cannabis. Species—sativa.

### VARIETY DENOMINATION

The new Cannabis plant claimed is of the variety denominated ‘DRG1’.

### BACKGROUND OF THE INVENTION

### Field of the Invention

The present invention relates to a new and distinct annual variety of C. sativa, which has been given the variety denomination of ‘DRG1’. ‘DRG1’ is intended for use as a medicinal herb for sale in Cannabis dispensaries.

### BACKGROUND OF THE RELATED ART

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense, legal commercialization in the United States as industrial hemp for a variety of purposes including biodegradable plastics and building materials, clothing, paper, food, fuel, and medicines.

Cannabidiol (CBD) extracted from Cannabis is widely used in over-the-counter medicines and topical treatments, and is also the active ingredient in the FDA-approved drug Epidiolex. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of C. sativa, which has been given the variety denomination of ‘DRG1’. ‘DRG1’ is intended for use as use as a medicinal herb for sale in Cannabis dispensaries.

The new C. sativa variety is a selection resulting from pollen breeding of ‘Pineapple’ female (not patented) from the mountains of northern Mendocino County, CA and an heirloom ‘Blackberry Kush’ (not patented) sourced from the interior highlands of Humboldt County, CA. The new variety was discovered and selected as a single plant within a population of 200+ resulting C. sativa hybrid plants from this controlled pollination in 2007 in an outdoor, full-sun garden at Mendocino County, CA. Selection criteria included isolation of a single female with superior traits from both parents and an uplifting effect that reduces pain.

Asexual reproduction of the new variety by cutting propagation since 2007 in Mendocino County, CA has demonstrated that the new variety reproduces true to type with all of the characteristics, as herein described, firmly fixed and retained through successive generations of such asexual propagation. A stable seed line of the new variety has also been developed by successive generations of interbreeding and selection to correspond to the phenotypic characteristics described herein.

The following characteristics of the new variety have been repeatedly observed and can be used to distinguish ‘DRG1’ as a new and distinct variety of C. sativa hybrid plant:
1. Dominant “fruit and berries” fragrance.
2. Purple bracts predominate.
3. Dense, elongated flower buds with tight intemodal span.
4. Long flower colas.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying photographic illustrations show the typical appearance of the new variety ‘DRG1’. The colors are as nearly true as is reasonably possible in a color representation of this type. Colors in the photographs may differ slightly from the color values cited in the detailed botanical description, which accurately describes the colors of the new plant.

FIG. 1 is a photograph of the new variety ‘DRG1’, at 58 days.

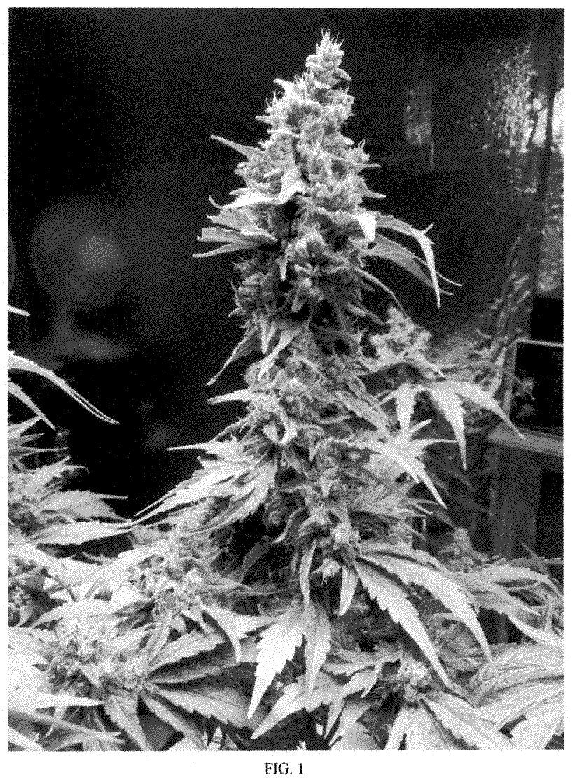

FIG. 2 is a photograph of the new variety ‘DRG1’ at 42 days grown in aeroponic conditions under light-emitting diode (LED) light.

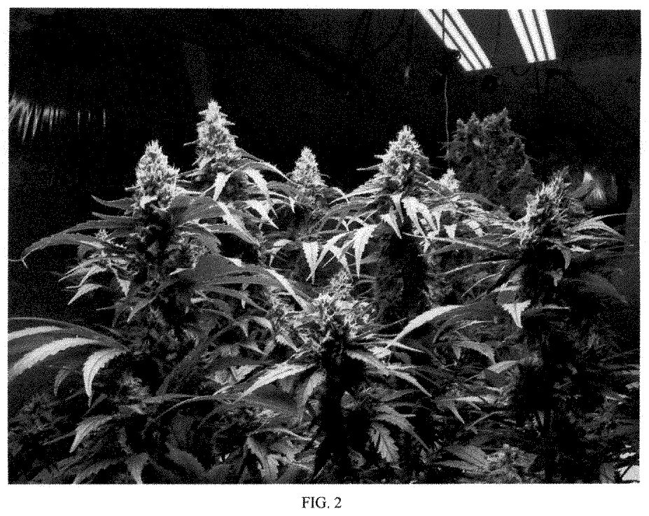

FIG. 3 is a photograph of the new variety ‘DRG1’ at 56 days grown indoors in aeroponic conditions.

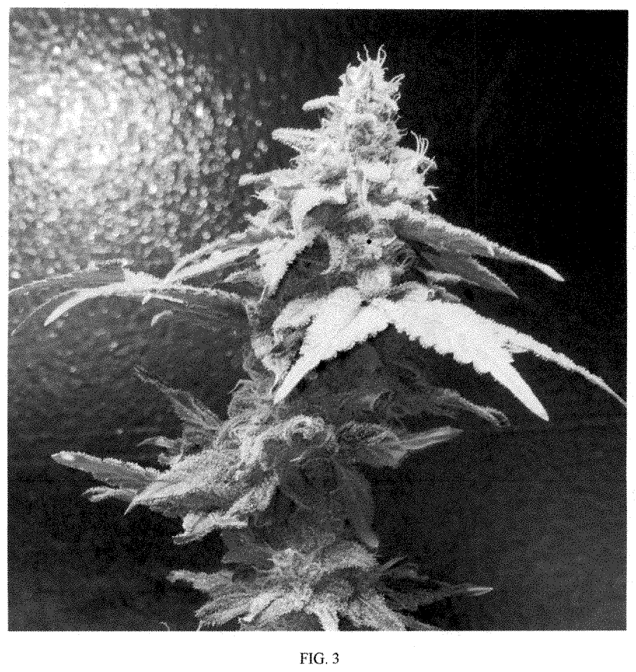

FIG. 4 is a photograph of the new variety ‘DRG1’ at 56 days indoor soil-less.

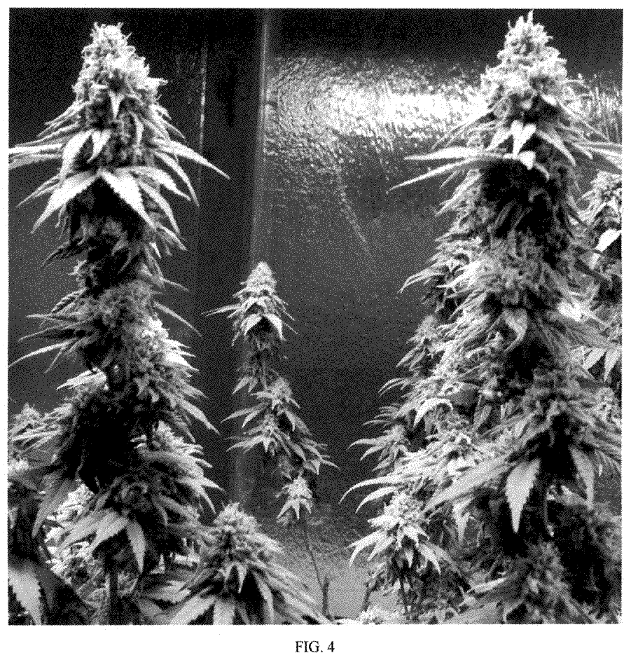

FIG. 5 is a photograph of the new variety ‘DRG1’ at 58 days grown indoors in soil-less conditions.

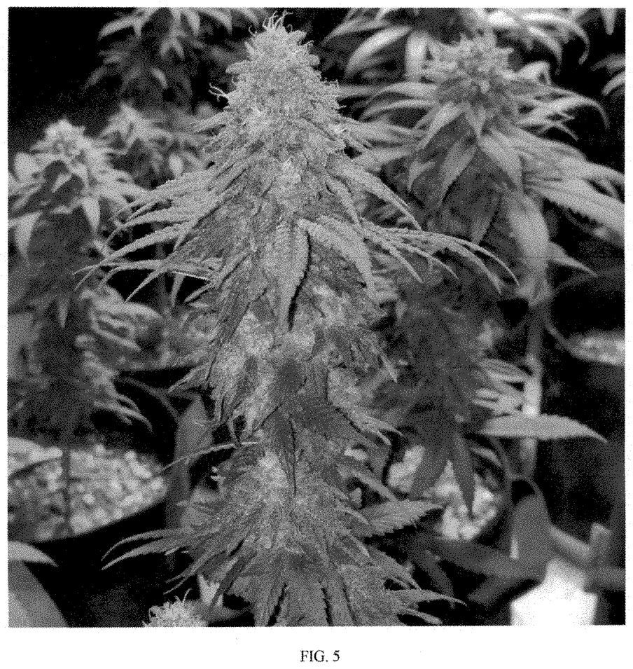

### DETAILED BOTANICAL DESCRIPTION

The following detailed description sets forth the distinctive characteristics of ‘DRG1’. The data which defines these characteristics was collected from asexual reproductions of the original selection. Dimensions, sizes, colors, and other characteristics are approximations and averages set forth as accurately as possible.
Type: Herbaceous tap-rooted annual.
   Classification.—
   a. Family.—Cannabaceae.
   b. Genus.—Cannabis.
   c. Species.—sativa.
   d. Common name.—Marijuana.
Plant:
   General.—.
   a. Origin.—Mendocino County, CA.
   b. Parentage.—Female ‘Pineapple’ from the mountains of northern Mendocino County, CA; Male: heirloom ‘Blackberry Kush’ sourced from the interior highlands of Humboldt County, CA.
   c. Growth habit.—Vigorous.
   d. Height.—150-200 cm.
   e. Plant spread.—150-180 cm.
   f. Growth rate.—Fast.
   g. Branching characteristics.—Tight and upswept.
   h. Length of primary lateral branches.—70-95 cm.
   i. Quantity of primary lateral branches.—>16.
   j. Characteristics of primary lateral branches.—i. Color — Green with purple veining. ii. Texture — Smooth with vertical ribbing. iii. Strength — Strong.
   k. Internode length.—Short and compact.
   l. Cold hardiness.—Hardy.
Foliage:
   General.—Dense and bushy.
   a. Leaf.—Broad and Flat.
   b. Arrangement.—Opposite decussate to alternate in flower.
   c. Quantity.—Varies by height.
   d. Leaf color (Top side).—Dark green to purple.
   e. Leaf color (Under side).—Light green.
   f. Leaf arrangement.—Opposite decussate emerging to alternate in flower.
   g. Leaf shape.—5-7 finger sessile leaflets.
   h. Leaf margins.—Serrate.
   i. Undulation of margin.—Typical per species.
   j. Leaf apices.—Pointed attenuate.
   k. Leaf bases.—Sagittate.
   l. Leaf width.—7 cm.
   m. Leaf length.—11-16 cm.
   n. Texture of top and bottom surfaces.—Medium veined.
   o. Appearance of top and bottom surfaces.—Fine veined.
   p. Venation type.—Palmate.
   q. Petiole length.—5 cm.
   r. Petiole color.—Green.
   s. Petiole texture.—Smooth and veined.
Inflorescence:
   General.—A complex pinnacle.
   a. Natural flowering season.—Mid-late September.
   b. Inflorescence and flower type and habit.—Main branch panicle.
   c. Fragrance.—Fruit and berries.
   d. Female flower and bud.—i. Bud shape — Urn panicle. ii. Bud length — 5 cm. iii. Bud diameter — 4.25 cm. iv. Bract diameter — 0.3 cm. v. Bract length — 0.3-0.5 cm. vi. Bract quantity per flower — Varies by size. vii. Bract shape — Ovate. viii. Bract base — Perigonal. ix. Bract texture — Hairy. x. Stigma length —¼-½ inch. xi. Stigma color — White to light pink. xii. Stigma number — 2. xiii. Stigma shape — Round hirsute. xiv. Ovary position — Bracts base. xv. Ovary shape — Ovate. xvi. Ovary length — About 1/32- 1/16 inch.
Seeds:
   General.—
   a. Shape.—Obtuse.
   b. Length.—About 0.4 cm.
   c. Width.—0.3 cm.

‘DRG1’ can have a cannabinoid profile as set forth in Table 1 and a terpene profile as set forth in Table 2. Due to the natural variability of chemotypic expression that is commonly observed in Cannabis plants, arising from numerous causes such as cultivation conditions, the values set forth in the tables do not reflect the only possible range of outcomes that can be obtained from plants of the new variety. Thus, these values are merely exemplary of observed values (middle column) and predicted, normal variations from the observed values.

TABLE 1
Exemplary Profiles of Key Cannabinoids.

| | Percent | Percent | Percent |
|---|---|---|---|
| THCa | 15.4736 | 19.342 | 23.2104 |
| CBGa | 2.352 | 2.94 | 3.528 |
| Δ9THC | 1.592 | 1.99 | 2.388 |
| CBCa | 0.704 | 0.88 | 1.056 |
| CBG | 0.136 | 0.17 | 0.204 |
| CBDa | 0.0664 | 0.083 | 0.0996 |
| CBC | 0.048 | 0.06 | 0.072 |
| THCVa | 0.0424 | 0.053 | 0.0636 |
| Total THC* | 15.1624 | 18.953 | 22.7436 |
| Total CBD** | 0.0584 | 0.073 | 0.0876 |
| Total | 18.1216 | 22.652 | 27.1824 |
| Cannabinoids*** | | | |

*Total THC = Δ9THC + (THCa * 0.877)
**Total CBD = CBD + (CBDa * 0.877)
***Total Cannabinoids = Total THC + Total CBD + Total CBG + Total THCV + Total CBC + Total CBDV + Δ8THC + CBL + CBN

TABLE 2
Exemplary Profiles of Key Terpenes.

| | Percent | Percent | Percent |
|---|---|---|---|
| Ocimeme | 0.392 | 0.49 | 0.588 |
| beta | 0.284 | 0.355 | 0.426 |
| Caryophyllene | | | |
| Limonene | 0.1056 | 0.132 | 0.1584 |
| alpha | 0.0736 | 0.092 | 0.1104 |
| Humulene | | | |
| alpha Pinene | 0.0624 | 0.078 | 0.0936 |
| alpha | 0.056 | 0.07 | 0.084 |
| Bisabolol | | | |
| beta Pinene | 0.032 | 0.04 | 0.048 |
| Linalool | 0.032 | 0.04 | 0.048 |
| Myrcene | 0.016 | 0.02 | 0.024 |
| Terpineol | 0.016 | 0.02 | 0.024 |
| Total | 1.0696 | 1.337 | 1.6044 |
| Terpenoids | | | |

1. A new and distinct variety of Cannabis plant named ‘DRG1’, as illustrated and described herein.
